(19) United States
(12) Patent Application Publication
(10) Pub. No.: US 2004/0166080 A1
(43) Pub. Date: Aug. 26, 2004

(54) Thermoplastic coating and binding agent for medicinal forms

(75) Inventors: Manfred Assmus, Bickenbach (DE); Hans-Ulrich Petereit, Darmstadt (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: ROEHM GBMH & CO. KG, Darmstadt (DE)

(21) Appl. No.: 10/788,269

(22) Filed: Mar. 1, 2004

Related U.S. Application Data
(62) Division of application No. 08/813,950, filed on Mar. 3, 1997.
Application No. 08/813,950 is a division of application No. 08/603,765, filed on Feb. 16, 1996, now abandoned.

(30) Foreign Application Priority Data
Feb. 16, 1995 (DE) G 295 02 547.6

(51) Int. Cl. A61K 7/06; A61K 7/11; A61K 9/20

(52) U.S. Cl. 424/70.16

(57) ABSTRACT

A thermoplastic coating and binding agent containing a non-homogeneous mixture of, based on the total weight of A and B:

A) 5-95 wt % of a thermoplastic acrylic plastic with a melting temperature above room temperature, but below 200° C., a glass transition temperature below 120° C., and a melt viscosity of 1,000 to 1,000,000 Pa·sec at the melting temperature; and

B) 95-5 wt. % of a flow improver, which, at room temperature, is not compatible with the thermoplastic acrylic plastic, has a melting temperature above room temperature but below 200° C., a molecular weight under 20,000 d, and a melt viscosity below 100 Pa·sec at the melting temperature of the acrylic plastic.

### FIELD OF THE INVENTION

The invention concerns a thermoplastic coating and binding agent. More particularly, the invention concerns a thermoplastic coating and binding agent for the preparation or sheathing from the melt-liquid state of active materials, including medicinal forms, in particular, of solid medicinal forms which can be administered orally.

### DESCRIPTION OF THE PRIOR ART

In addition to the classical method for the preparation of solid medicinal forms by the pressing of tablets and sheathing with a coating agent dissolved or dispersed in a liquid phase, the preparation or sheathing of medicinal forms from the melt-liquid state of the coating and binding agent is gaining increasing importance as a particularly economical and reliable preparation method. This requires thermoplastic coating and binding agents, which must fulfill a number of special requirements:

they must fulfill the dissolution or release requirements needed for the use of the medicinal form;

they must be meltable, undecomposed, and be capable of mixing in the solid state by cooling;

they must produce a dry, nonsticky surface upon solidification from the melt;

they should not damage the contained or sheathed pharmaceutical active substance under the conditions of thermoplastic processing.

The thermoplastic coating and binding agents available at present do not meet these requirements to a satisfactory extent. This is true, above all, for coating and binding agents on the basis of acrylic plastics, which are generally known under the tradename EUDRAGIT from the Röhm GmbH, Darmstadt. They are characterized by special solubility and release characteristics, which cannot be dispensed with for the preparation of delayed action preparations. Thus, EUDRAGIT E contains basic amino groups, which ensure the solubility in gastric juice. EUDRAGIT RL and RS contain quaternary ammonium groups, which control the active substance release independent of the pH value of the surrounding aqueous medium.

N. Follonier, E. Doelker, and E. Cole (Drug Development and Industrial Pharmacy, 20(8), 1323-1339, 1994) describe the thermoplastic processing of EUDRAGIT RS PM with an addition of 3-5% triacetin as a plasticizer and diltiazem HCl as an active substance at a temperature of 110° C. Medicinal forms with a macro- and microporous surface are obtained, from which the active substance was released at a rate clearly higher than from medicinal forms with a traditional coating of EUDRAGIT RS.

The preparation of medicinal forms by extrusion and rounding out, using thermoplastic coating and binding agents based on acrylic plastics has already been described. If aqueous dispersions or moist mixtures of these coating and binding agents are used with such methods and a drying step is used, they do not correspond to the goal of the thermoplastic preparation of medicinal forms discussed here.

According to German Patent Application No. A 4,138,513, mixtures of a pharmaceutical active substance and a thermoplastic binder are extruded from the melt and processed to medicinal forms. Mixtures of EUDRAGIT RL or RS with vinylpyrrolidone-vinyl acetate copolymers and hydroxypropylcellulose are used as thermoplastic binders. The same release characteristics cannot be attained in this way as with the corresponding acrylic plastics or their mixtures alone.

According to European Patent Application No. A 204,596, active-substance-containing microparticles of a mixture which contain as a binder a polymer in a mixture with one or more excipients are extruded. The excipients are selected in such a way that, individually or jointly, they exert a dissolving or gelling effect and a lubricating effect on the polymer. During the processing by means of perforated rolls, the mixture is heated, so that one of the excipients melts in part and thereby evolves a dissolving or gelling effect. The mixture, which is plasticized in this way, is pressed through the openings of the perforated rolls and thereby extruded to microparticles. As an example, the processing of powdery EUDRAGIT RS with a glycerol palmitostearate is described.

The processing of a genuine melt of this mixture would not lead to a usable medicinal form. A homogeneous mixture would form from the polymer and the excipient; it would not segregate once again upon cooling because of the presupposed dissolving or gelling effect. The consequence of the plasticizing effect of the excipient is that the composition solidified from the melt remains soft and sticky on its surface, so that it would not be usable as a medicinal form surface.

A thermoplastic coating and binding agent, based on an acrylic and/or methacrylic acid-methyl acrylate copolymer, is known from German Utility No. Model G 9,414,065. It is suitable for the production of gastric-juice-soluble medicine sheaths from the melt at temperatures of 120-180° C., but not for the purposes for which EUDRAGIT RL and RS are used. Plasticizers can be used without making the surface sticky because of the high melting temperature of the copolymer.

### OBJECT OF THE INVENTION

The object of the invention is to make available a thermoplastic coating and binding agent, which fulfills the requirements described above. Release characteristics should be attained which correspond approximately to those of traditional film coatings made of aqueous dispersions or organic solutions. The prerequisite for this is an improved flowing capacity of the melt, without a plasticizing effect, however, which would lead to sticky surfaces.

### SUMMARY OF THE INVENTION

The object of the invention is achieved by a thermoplastic coating and binding agent, containing a non-homogeneous mixture of, based on the total weight of A and B:

A) 5-95 wt % of a thermoplastic acrylic plastic with a melting temperature above room temperature, but below 200° C., a glass transition temperature below 120° C., and a melt viscosity of 1,000 to 1,000,000 Pa·sec, preferably 10,000 to 500,000 Pa·sec, at the melting temperature; and

B) 95-5 wt % of a flow improver, which, at room temperature, is not compatible with the thermoplastic acrylic plastic, has a melting temperature above room temperature but below 200° C., a molecular weight under 20,000 d, and a melt viscosity below 100 Pa·sec, at the melting temperature of the acrylic plastic.

### DETAILED DESCRIPTION OF THE INVENTION

The melting temperature in the sense of the invention is considered to be the lowest temperature at which the melt viscosity, measured according to DIN 54811, exceeds the limiting value of 10⁶ Pa·sec. The glass transition temperature is thermally determined by means of DSC (Differential Scanning Calorimetry). The molecular weight is considered to be the weight average, determined by gel permeation chromatography (GPC). If a corresponding calibration curve is set up, the determination of the molecular weight from the reduced dissolution viscosity is simpler.

The mixture of the components A and B, in accordance with the invention, is regarded as non-homogeneous, if the components A and B are not compatible in the quantities used. This can be recognized on a film which is left behind from a common solution of the components in a common solvent, such as acetone, during the evaporation of the solvent. If the film is clearly murky, then this allows us to deduce the presence of two phases which are not compatible with one another. With limited compatibility, two phases may form, in which one of the components predominates proportionately. Such mixtures are considered incompatible in the sense of the invention if the glass transition temperature of the mixture does not lie substantially, in particular no more than 20° K., below the glass transition temperature of polymer A.

The incompatibility has the effect that in the solidified melt, components A and B are present as separate phases, and flow improver B is not present dissolved in polymer phase A as a plasticizer. The phases may be present in domains of a small size; preferably, component B forms separate, disperse phases of 0.1 to 500 μm in size within cohesive phase A. The quantitative proportion of phases A:B is preferably selected in such a way that A forms a cohesive phase. The phase size can be recognized in the light or electron microscopic image.

The presence of two phases can be detected also by differential thermoanalysis (DSC measurement). Incompatible mixtures are characterized by two thermal signals, which essentially correspond to those of components A and B.

Components A and B are essentially miscible in the melt state, recognizable in the optical clarity of the melt. A good flowing capacity and a melt viscosity of less than 500 Pa·sec, preferably 1 to 200 Pa·sec, result from the compatibility in the melt state. In this way, a rapid plasticizing, a low processing temperature, low shear forces during processing, connected with a low thermal decomposition, a complete and precise filling of mold tools and a closed, low-pore surface of the solidified melt are attained. The release behavior is mainly determined by the characteristics of polymer component A and to a large extent corresponds to that of a corresponding medicinal form with a jacket produced from the organic solution of the same polymer A.

Thermoplastic acrylic plastics are understood to mean uncrosslinked, optionally branched, polymers and copolymers, which, in any case, are at least colloidally soluble in suitable organic solvents, such as acetone, isopropyl alcohol or ethanol, which are synthesized by at least 20 wt %, preferably at least 50 wt %, from acrylic monomers. These monomers are characterized by the group:

CH₂═CR—CO—

wherein R represents either a hydrogen atom or a methyl group. Their molecular weight is preferably 10,000 to 200,000 d; their melt viscosity at 100° C., preferably 10,000 to 500,000 Pa·sec. A glass transition temperature above room temperature and below 120° C. is of essential importance; the preferred range is 30 to 80° C.

The thermoplastic acrylic plastics are generally copolymers of esters of acrylic and/or methacrylic acid, in particular copolymers of alkyl esters of the acrylic and/or methacrylic acid and functional comonomers with covalently bound cationic groups. Preferably, the alkyl esters make up 5 to 99 wt % of the copolymer, and the cationic comonomers, 1 to 95 wt %. Other comonomers can also be used, including hydroxyalkyl esters or alkoxyalkyl esters of the acrylic and/or methacrylic acid or small quantities of this acid itself, derivatives of maleic acid, styrene, vinyl acetate. As a rule, they make up no more than 40 wt % of the copolymer.

The acrylic esters of the acrylic and/or methacrylic acid are preferably derived from lower alkanols, in particular those with 1 to 4 carbon atoms in the alkyl radical. Methyl acrylate and methacrylate and ethyl acrylate and methacrylate are particularly preferred.

The cationic comonomers may be aminoalkyl esters or aminoalkylamides of acrylic and/or methacrylic acid or their salts or quaternary products. Preferably, they contain tertiary amino or quaternary ammonium groups, which are connected to the ester oxygen atom or the amide nitrogen atom by means of a lower alkyl radical, preferably with 1 to 5 carbon atoms. As a rule, they can be represented by the following formula:

CH₂═CR—CO—X—Alk-N(R′)₂

or

CH₂═CR—CO—X—Alk-N+(R′)₃ An⁻

wherein R is H or CH₃—, R′ is C₁₋₄ alkyl; X is the group -0- or —NH—; Alk is a straight-chain or branched alkyl radical with 1 to 5 carbon atoms; and An- is a monovalent acid anion.

Suitable cationic comonomers are, for example, the following:

2-(N,N-dimethylamino)ethyl acrylate and methacrylate;

3-(N,N-dimethylamino)propyl acrylate and methacrylate;

4-(N,N-dimethylamino)butyl acrylate and methacrylate;

3-(N,N-dimethylamino)propylacrylamide and -methacrylamide;

triethanolamine monoacrylate and monomethacrylate;

2-(dimethylaminoethyloxy)ethyl acrylate and methacrylate;

2-imidazolylethyl acrylate and methacrylate;

2-piperazinylethyl acrylate and methacrylate;

2-piperazinylethylacrylamide and -methacrylamide;

N,N-dimethylaminoneopentyl acrylate and methacrylate;

N,N-dimethylaminoneopentylacrylamide and -methacrylamide;

(1,2,2,6,6-pentamethyl-4-piperidyl) acrylate and methacrylate; 3-morpholinopropylacrylamide and -methacrylamide;

2-morpholinoethyl acrylate and methacrylate;

2-(N,N-dibutylamino)ethyl acrylate and methacrylate;

4-diethylamino-1-methylbutylacrylamide and -methacrylamide;

and the quaternary products which can be produced therefrom with methyl chloride or other quaternary agents and the salts of the so-called monomers with organic or inorganic acids, such as hydrochloric acid, sulfuric acid, phosphoric acid, acetic acid, propionic acid, salicylic acid, succinic acid, lauric acid, and so forth.

In actual practice, copolymers of methyl acrylate and methacrylate and/or ethyl acrylate and methacrylate with 1-95 wt % dimethylaminoethyl acrylate and methacrylate or trimethyammonioethyl acrylate chloride and methacrylate chloride play the most important role. Among this group belong EUDRAGIT RL, RS AND E.

The polymers used in accordance with the invention must have a purity suitable for pharmaceutical use if used as such. In particular, the residual monomer content should be below 1,000 ppm.

Flow improver B includes substances that can be mixed in an essentially homogeneous manner with the melt of polymer A and improve the flowability of the melt, but can be separated when the melt is cooled and solidified as its own phase. Substances that fulfill these prerequisites have a clearly lower molecular weight than polymer A and a generally lower polarity deviating therefrom. The polarity is determined by the fraction of hydrophilic and hydrophobic groups in the molecular structure of the flow improver. oxygen atoms in the form of hydroxy, ether, ester, and carbonyl groups and nitrogen atoms in the form of amino, ammonium and amide groups, which can occur in cyclic molecule structures also, increase the polarity. On the other hand, the polarity is reduced by longer aliphatic or olefinic radicals, in particular those with 6 to 30 carbon atoms, and by aromatic radicals.

If the polarity of the flow improver is excessively high, then its dissolving and plasticizing characteristics predominate. They prevent the phase separation during solidification and lead to a sticky surface. If the polarity is too low, there is the danger that the mixture of A and B also remains diphasic in the melt. Then, the desired improvement of the flowability may not occur or there may even be segregation processes, which can be detrimental during extrusion. It is simpler to determine the compatibility in the melt state and the incompatibility in the solidified state by preliminary experiments than to calculate them with the aid of polarity increments. The selection of a suitable flow improver can be facilitated sometimes if mixtures of different substances, in particular those of different polarities, are prepared, and the best-suited total polarity is determined by a variation of the mixing proportions.

The compatibility characteristics are, however, also influenced by the molecular weight of the flow improver. In general, the compatibility declines, with otherwise constant molecular structure, with rising molecular weight. Thus, for example, polyethylene glycols with molecular weights below 1,000 d are mostly less suited as flow improvers than those with molecular weights of 1,000 to 20,000 d. With the somewhat less polar polypropylene glycols, the molecular weight limit is lower. As a rule, substances with molecular weights above 20,000 d are unsuitable, since they do not reduce the melt viscosity to the desired extent.

Suitable flow improvers are found in various substance classes, for example, under fatty alcohols, fatty acids, surfactants, mono-, di- and triglycerides. A melting temperature above room temperature but below 200° C., preferably in the range of 30 to 150, in particular 40 to 80° C., is important.

For copolymers of lower alkyl acrylates and methacrylates with 5 to 10 wt % cationic amino- or ammonium-group-containing comonomers of the EUDRAGIT RL, RS, and E type, fatty acid monoglycerides, in particular glycerol monostearate and polyethylene glycols with molecular weights of 4,000 to 8,000 d, have proved very suitable.

As examples of other flow improvers, one can mention the following:

fatty acids, such as

stearic acid,

lauric acid,

palmitic acid,

capric acid,

myristic acid;

sugars, such as sorbitol;

waxes, such as

beeswax,

wool wax,

spermaceti;

fatty alcohols, such as

cetyl alcohol,

stearyl alcohol;

polyethylene glycols, such as

PEG 1000,

PEG 2000,

PEG 6000;

glycerol esters, such as

glycerol laurate,

glycerol monostearate,

glycerol distearate,

glycerol tristearate;

fatty acid fatty alcohol esters, such as

palmitic acid cetyl ester;

sugar esters, such as

sorbitan monostearate;

polyethylene glycol fatty acid esters, such as

polyethylene glycol 400 stearate;

polyethylene glycol fatty alcohol ethers, such as

polyethylene glycol 23 lauryl ether.

Likewise, mixtures of flow improvers of the aforementioned substance classes are suitable if they exhibit the characteristics required for the flow improver as a mixture.

The quantity of the flow improver or the mixing ratio A:B is oriented on the flow-improving effectiveness and thus also on the requirements of the processing method. In general, a melt viscosity of the thermoplastic coating and binding agent of less than 4,000 Pa·sec, preferably below 1,000 Pa·sec, in particular below 500 Pa·sec, is desired, measured at 100° C. In many cases, melt viscosities of 50 to 250 Pa·s are attainable. The fraction of the flow improver is then, as a rule, between 10 and 60 wt %, preferably 20 to 50 wt %, based on the total weight of A and B.

An influence of the flow improver on the release characteristics in comparison to the pure polymer component A should not be ruled out and must, if necessary, be taken into consideration in putting the mixture together. Thus, carboxylic acids, as flow improvers, can promote the dissolution or permeability of the thermoplastic coating and binding agent in the gastric juice with increasing pH value.

The thermoplastic coating and binding agent in accordance with the invention are appropriately prepared in the desired mixing ratio. To this end, components A and B and, if desired, other additives common in medicine coatings, such as fillers, pigments, dyes, dispersants, stabilizers, aromas, are uniformly mixed in the melt, cooled, and after solidification, comminuted to a powder or granular material. In particular, anionic polymers which do not melt under the indicated conditions can be used for the modification of the release behavior. A mixing extruder is used advantageously, wherein polymer component A, as a powder or granular material, is preferably introduced as a mixture with the flow improver B, melted and homogenized. The mixing temperature is, e.g., 100-150° C. The mixture is discharged from the extruder as a strand and granulated by hot fragmenting or by breaking after cooling. Heatable kneaders can also be used.

The medications used in the sense of the invention are intended to be used on or in the human or animal body, in order:

1. to heal, ameliorate, prevent or to recognize diseases, suffering, body injury, or pathological complaints;

2. to permit a recognition of the nature, the state, or the functions of the body or mental condition;

3. to replace active substances or body fluid produced by the human or animal body;

4. to fend off, eliminate, or render harmless pathogens, parasites, or substances alien to the body; or

5. to influence the nature, the state, or the functions of the body or mental condition.

Common medications can be found in reference works, such as the Red List or the Merck Index. In accordance with the invention, all active substances are used which fulfill the desired therapeutic effect in the sense of the definition above and have a sufficient thermal stability.

Important examples (groups and individual substances) are the following:

antiallergic agents, antiarrhythmic drugs, antibiotics/chemotherapeutics, antidiabetics, antiepileptics, antihypertensive agents, antihypnotics, anticoagulants, antimycotic agents, antiphlogistics, beta-receptor blockers, calcium antagonists, ACE inhibitors, bronchyltics/antiasthmatics, corticoids (internal), dermatic agents, diuretics, enzyme inhibitors, enzyme preparations, and transport proteins, geriatric medicines, gout remedies, influenza medicines, hypnotics/sedatives, cardiac stimulants, lipid lowering agents, parathyroid hormones/calcium metabolism regulators, psychopharmacological agents, sexual hormones and their inhibitors, spasmolysants, agents for the treatment of wounds, cytostatic drugs.

For greater productions of medicinal forms, it may be appropriate to work into the mixture one or more pharmaceutical active substances already during the production of the mixture while hot. The granular material obtained can be poured into capsules immediately or perhaps, with the addition of tableting auxiliaries, can be pressed to tablets.

Methods for the thermoplastic preparation of solid medicinal forms are known. Among these are the injection molding processes, which, however, were regarded as not very suitable for a long period of time, because they yielded casting lugs during the production of injection-molded tablets, which, because of their content of valuable active substances, could not be simply discarded or disposed of. There was concern against a renewed thermoplastic processing because uncontrollable decompositions at high processing temperatures and under high shear forces were expected. The invention now makes possible rather low processing temperatures and a high flowability, so that the thermal and rheological stress of the material is limited and lugs of low volume are sufficient. Therefore, in the individual case, it must be tested whether tablet production in the injection molding process is justifiable.

In general, the production of medicinal forms by the extrusion of a strand is preferred. Dosage units can be separated by hot fragmenting, which can still be rounded off before solidifying with mechanical means or in a warm air whirlpool.

Flat medicinal forms, such as transdermally acting plasters, can be produced by the extrusion of films, perhaps on flat carriers.

Active-substance-free coating compositions can be used for injecting around pressed tablets. At the beginning of the injection process, they can be held in the center of a mold cavity by means of support props and be enclosed by the melt. Before solidification, the support props are withdrawn and the sheathing of the tablet core is completed.

Low-viscous melts can be sprayed onto tablet and dragee cores in compulsory mixers or fluidized bed units. Appropriately, the coated medicinal forms are allowed to pass from a warm air fluidized bed into a cold air fluidized bed for cooling; the coated medicinal forms are removed from there. The usual layer thicknesses, for example, 10 to 200 μm, are attained. A suitable coating process was described by M. J. Jozwiakowski et al. in Pharmaceutical Research, Vol. 7, November 1990, pp. 3-10.

As a rule, the medicinal forms prepared in accordance with the invention exhibit release behavior of the contained active substance in gastric or intestinal juices, which is typical of the polymer component. Medicinal forms with a matrix or a coating based on a polymer containing amino groups, such as EUDRAGIT E, are dissolved in artificial gastric juice within a maximum of 2 hours and release the active substance. Polymers with quaternary ammonium groups, such as EUDRAGIT RL or RS, produce coatings or matrices whose solubility and diffusion permeability is independent of the pH value of the surrounding aqueous medium. The medicinal forms remain undissolved in artificial gastric and intestinal juices and gradually release the active substance by diffusion as a function of the ammonium group content. By a mixture of EUDRAGIT RL AND RS, intermediate values of the release rate can be established.

Having generally described this invention, a further understanding can be obtained by reference to certain specific examples which are provided herein for purposes of illustration only and are not intended to be limiting unless otherwise specified.

### EXAMPLE 1

500 g EUDRAGIT RS 100 are mixed with 250 g glycerol monostearate in a heatable kneader at 120 degrees C. The composition formed is whitish, solid and homogeneous at room temperature. The glass transition temperature (DSC) is 50° C.; the melt viscosity at 100° C. is below 100 Pa·sec.

### EXAMPLE 2

500 g EUDRAGIT RS 100 are mixed with 250 g polyethylene glycol 6000 in a heatable kneader at 120° C. The composition formed is whitish, solid and homogeneous at room temperature.

The glass transition temperature (DSC) is 50° C.; the melt viscosity at 100° C. is 221 Pa·sec.

### EXAMPLE 3

500 g EUDRAGIT RL 100 are mixed with 250 g polyethylene glycol 6000 in a heatable kneader at 120° C. The composition formed is whitish, solid and homogeneous at room temperature.

The glass transition temperature (DSC) is 55° C.; the melt viscosity at 100° C. is 2858 Pa·sec.

### EXAMPLE 4

400 g EUDRAGIT E 100 are mixed with 300 g glycerol monostearate in a heatable kneader at 120° C. The composition formed is whitish, solid and homogeneous at room temperature.

The glass transition temperature (DSC) is 50° C.; the melt viscosity at 100° C. is below 100 Pa·sec.

### EXAMPLE 5

35 g methionine are worked into 350 g of the composition prepared in Example 4. The composition formed is whitish, solid and homogeneous at room temperature. The glass transition temperature (DSC) is 50° C.; the melt viscosity at 100° C. is below 100 Pa·sec. The release behavior of the active substance is controlled by the polymer. Obviously, numerous modifications and variations of the present invention are possible in light of the above teachings. It is therefore to be understood that within the scope of the appended claims, the invention may be practiced otherwise than as specifically described herein.

What is claimed as new and desired to be secured by Letters Patent of the United States is:

1. A thermoplastic coating and binding agent containing a non-homogeneous mixture of, based on 100% by weight of A and B:
A) 5-95% of a thermoplastic acrylic plastic with a melting temperature above room temperature and below 200° C., a glass transition temperature below 120° C., and a melt viscosity of 1,000 to 1,000,000 Pa·sec at the melting temperature; and
B) 95-5 wt % of a flow improver, which, at room temperature, is not compatible with the thermoplastic acrylic plastic, has a melting temperature above room temperature but below 200° C., a molecular weight under 20,000 d, and a melt viscosity below 100 Pa·sec at the melting temperature of the acrylic plastic.

2. The thermoplastic coating and binding agent according to claim 1, which is solidified from a melt thereof.

3. The thermoplastic coating and binding agent according to claim 1, wherein the thermoplastic acrylic plastic A is a copolymer of esters of acrylic and/or methacrylic acid.

4. The thermoplastic coating and binding agent according to claim 2, wherein the thermoplastic acrylic plastic A is a copolymer of esters of acrylic and/or methacrylic acid.

5. The thermoplastic coating and binding agent according to claim 3, wherein the thermoplastic acrylic plastic A is a copolymer of alkyl esters of acrylic and/or methacrylic acid and functional comonomers with covalently bound cationic groups.

6. The thermoplastic coating and binding agent according to claim 4, wherein the thermoplastic acrylic plastic A is a copolymer of alkyl esters of acrylic and/or methacrylic acid and functional comonomers with covalently bound cationic groups.

7. The thermoplastic coating and binding agent according to claim 5, wherein the thermoplastic acrylic plastic A is a copolymer of 5 to 99 wt % alkyl esters of acrylic and/or methacrylic acid and 95 to 1 wt % aminoalkyl esters or aminoalkylamides of acrylic and/or methacrylic acid or their salts or quaternary ammonium compounds thereof.

8. The thermoplastic coating and binding agent according to claim 6, wherein the thermoplastic acrylic plastic A is a copolymer of 5 to 99 wt % alkyl esters of acrylic and/or methacrylic acid and 95 to 1 wt % aminoalkyl esters or aminoalkylamides of acrylic and/or methacrylic acid or their salts or quaternary ammonium compounds thereof.

9. The thermoplastic coating and binding agent according to claim 1, wherein flow improver B is a fatty alcohol, a fatty acid, an ester of of a fatty alcohol and a fatty acid, a sugar, an ester thereof, a fatty acid mono-, di-, or triglyceride, a polyethylene glycol, a fatty acid ester or fatty alcohol ether thereof, a wax, or mixtures of any of the above.

10. The thermoplastic coating and binding agent according to claim 2, wherein flow improver B is a fatty alcohol, a fatty acid, an ester of of a fatty alcohol and a fatty acid, a sugar, an ester thereof, a fatty acid mono-, di-, or triglyceride, a polyethylene glycol, a fatty acid ester or fatty alcohol ether thereof, a wax, or mixtures of any of the above.

11. The thermoplastic coating and binding agent according to claim 3, wherein flow improver B is a fatty alcohol, a fatty acid, an ester of of a fatty alcohol and a fatty acid, a sugar, an ester thereof, a fatty acid mono-, di-, or triglyceride, a polyethylene glycol, a fatty acid ester or fatty alcohol ether thereof, a wax, or mixtures of any of the above.

12. The thermoplastic coating and binding agent according to claim 4, wherein flow improver B is a fatty alcohol, a fatty acid, an ester of of a fatty alcohol and a fatty acid, a sugar, an ester thereof, a fatty acid mono-, di-, or triglyceride, a polyethylene glycol, a fatty acid ester or fatty alcohol ether thereof, a wax, or mixtures of any of the above.

13. The thermoplastic coating and binding agent according to claim 5, wherein flow improver B is a fatty alcohol, a fatty acid, an ester of of a fatty alcohol and a fatty acid, a sugar, an ester thereof, a fatty acid mono-, di-, or triglyceride, a polyethylene glycol, a fatty acid ester or fatty alcohol ether thereof, a wax, or mixtures of any of the above.

14. The thermoplastic coating and binding agent according to claim 6, wherein flow improver B is a fatty alcohol, a fatty acid, an ester of of a fatty alcohol and a fatty acid, a sugar, an ester thereof, a fatty acid mono-, di-, or triglyceride, a polyethylene glycol, a fatty acid ester or fatty alcohol ether thereof, a wax, or mixtures of any of the above.

15. The thermoplastic coating and binding agent according to claim 7, wherein flow improver B is a fatty alcohol, a fatty acid, an ester of of a fatty alcohol and a fatty acid, a sugar, an ester thereof, a fatty acid mono-, di-, or triglyceride, a polyethylene glycol, a fatty acid ester or fatty alcohol ether thereof, a wax, or mixtures of any of the above.

16. The thermoplastic coating and binding agent according to claim 8, wherein flow improver B is a fatty alcohol, a fatty acid, an ester of of a fatty alcohol and a fatty acid, a sugar, an ester thereof, a fatty acid mono-, di-, or triglyceride, a polyethylene glycol, a fatty acid ester or fatty alcohol ether thereof, a wax, or mixtures of any of the above.

17. An oral or dermal medicinal composition containing a pharmaceutical active substance and the thermoplastic coating and binding agent of claim 2.

18. An oral or dermal medicinal composition containing a pharmaceutical active substance and the thermoplastic coating and binding agent of claim 4.

19. An oral or dermal medicinal composition containing a pharmaceutical active substance and the thermoplastic coating and binding agent of claim 6.

20. An oral or dermal medicinal composition containing a pharmaceutical active substance and the thermoplastic coating and binding agent of claim 10.
